(19) United States
(12) Patent Application Publication
(10) Pub. No.: US 2007/0254939 A1
(43) Pub. Date: Nov. 1, 2007

(54) Formulations Containing Amide Derivatives of Carboxylic Acid NSAIDS for Topical Administration to the Eye

(75) Inventor: Gustav Graff, Cleburne, TX (US)

Correspondence Address: ALCON, IP LEGAL, TB4-8, 6201 SOUTH FREEWAY, FORT WORTH, TX, 76134, US

(73) Assignee: ALCON, INC., Hunenberg (CH)

(21) Appl. No.: 11/740,379

(22) Filed: Apr. 26, 2007

Related U.S. Application Data
(60) Provisional application No. 60/795,908, filed on Apr. 28, 2006.

(51) Int. Cl. A61K 31/407; A61K 31/165

(52) U.S. Cl. 514/412; 514/620; 514/617

(57) ABSTRACT

Topical compositions of amide derivatives of carboxylic acid non-steroidal anti-inflammatory agents are disclosed. The compositions have a reduced potential to cause mitochondrial swelling when topically administered to the eye.

This application claims priority to U.S. Provisional Application, U.S. Ser. No. 60/795,908 filed Apr. 28, 2006.

### BACKGROUND OF THE INVENTION

This invention relates to topically administrable ophthalmic formulations of amide derivatives of carboxylic acid nonsteroidal anti-inflammatory agents (“NSAID's”). The formulations of the present invention are useful for treating ophthalmic inflammatory disorders.

Many non-steroidal anti-inflammatory agents (“NSAID's”) are known. One known class of NSAID's are carboxylic acid NSAIDs. Among other uses, carboxylic acid NSAID's are commonly used in connection with cataract surgery.

Despite improvements in surgical procedures and instrumentation, there continues to be a low incidence of corneal ulceration following cataract surgery with the use of topical NSAID's. Recent scientific literature indicates that NSAID's initiate programmed cell death (apoptosis) when used at concentrations higher than those needed for the inhibition of cyclooxygenase (i.e., prostaglandin synthesis). See, for example, See, for example, Zhang, et al., Leukemia Research, 24: 385-392 (2000); Taib, et al., Saudi Medical Journal, 25(10): 1360-1365 (2004); and Gomez-Lechon, et al., Biochemical Pharmacology, 66: 2155-2167 (2003). Apotosis is initiated by a free radical mechanism that causes mitochondria to swell and to release cytochrome c. Upon release, cytochrome c activates a serine protease (caspase-9) that promotes activation of other caspases, which cause subsequent degradation of nuclear components. What are needed are topical formulations of carboxylic acid NSAID's that minimize, prevent or eliminate the swelling response and release of cytochrome c from mitochondria, thereby reducing or eliminating side effects encountered with the topical use of carboxylic acid NSAID's.

U.S. Pat. No. 4,910,225 discloses topical formulations of certain carboxylic acid NSAID's that comprise a sulfite and a water-soluble polymer for enhanced stability. The concentration of the optional sulfite additive in the compositions of the '225 patent is “in the range of about 0.1 to 1.0 w/w %” (Col. 3, lines 61-62 of the '225 patent). The '225 patent does not suggest carboxylic acid NSAID compositions containing less than about 0.1 w/w % of sulfite or any compositions containing amide derivatives of carboxylic acid NSAID's.

U.S. Pat. No. 5,475,034 discloses topical formulations of certain amide derivatives of arylacetic acids. None of the compositions disclosed in the '034 patent contains a sulfite additive.

### SUMMARY OF THE INVENTION

The compositions of the present invention are topically administrable ophthalmic compositions containing an amide derivative of a carboxylic acid NSAID in an anti-inflammatory effective amount. In addition, the compositions comprise a sulfite salt in an amount effective to attenuate or prevent mitochondria swelling and cytochrome c release. The compositions also comprise an ophthalmically acceptable vehicle. The present invention also relates to methods of treating ophthalmic inflammatory disorders in mammals in need thereof. In one embodiment of the present invention, the compositions comprising an amide derivative of a carboxylic acid NSAID in an anti-inflammatory effective amount, a sulfite in an amount effective to attenuate or prevent mitochondria swelling and cytochrome c release, and an ophthalmically acceptable vehicle are topically administered to the mammal's eye. In another embodiment, separate compositions comprising a sulfite salt and an amide derivative of a carboxylic acid NSAID, respectively, are sequentially administered to the mammal's eye.

Among other factors, the present invention is based on the finding that mitochondria, when stressed by free radicals (such as hydroxyl free radicals, superoxide and peroxide), become sensitized to carboxylic acid NSAID's, including carboxylic acid NSAID's formed as metabolites of amide derivatives of carboxylic acid NSAID's. and swell. Mitochondrial swelling (i.e. opening of the permeability transition pore) is associated with the release of cytochrome c and initiation of the apoptotic pathway. This morphological change is induced through the opening of the mitochondrial permeability transition pore. Mitochondrial transition pore inhibitors are capable of preventing corneal ulceration induced in inflamed, peroxide-stressed tissue with exposure to NSAID's by preventing mitochondrial swelling, cytochrome c release, and subsequent apoptosis of corneal keratocytes. The latter cells are essential for corneal healing by producing cytokines and growth factors including synthesis of extracellular matrix components needed for tissue or wound repair.

### BRIEF DESCRIPTION OF THE DRAWINGS

FIG. 1 shows the time course of the in vitro swelling response of non-peroxide stressed (control) mitochondria following addition of carboxylic acid NSAID's.

FIG. 2 shows the time course of the in vitro swelling response of mitochondria following addition of t-BOOH (150 μM), t-BOOH/diclofenac (150 μM/30 μM), t-BOOH/diclofenac (150 μM/100 μM), or t-BOOH/diclofenac (150 μM/300 μM).

FIG. 3 shows the time course of the in vitro swelling response of peroxide (t-BOOH, 150 μM)-stressed mitochondria following addition of a) 60 μM amfenac; b) 60 μM bromfenac; c) 300 μM sodium sulfite/60 μM amfenac; d) 300 μM sodium sulfite/60 μM bromfenac; or e) nothing (negative control); CaCl₂ (positive control).

### DETAILED DESCRIPTION OF THE INVENTION

Unless indicated otherwise, all ingredient concentrations are presented in units of % weight/volume (% w/v).

The amide derivatives of carboxylic acid NSAID's suitable for use in the compositions and methods of the present invention are those of formulas (I), (II), and (III):

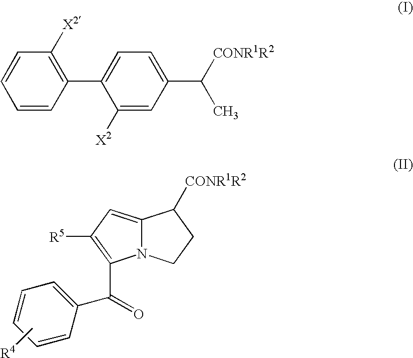

wherein for both formulas (I) and (II)

### R¹=H, C₁₋₆ (un)branched alkyl, (un)substituted (substitution as defined by Z below), —(CH₂)ₙ—X—(CH₂)ₙ.A;

### R²=H, C₁₋₃ alkyl, OR³;

### R³=H, C₁₋₃ alkyl;

### R⁴=H, Me-, MeO—, MeS—;

### R⁵=H, Me-;

### X=nothing (carbon-carbon bond), O, C═O, OC(═O), C(═O)O, C(═O)NR³, NR³C(═O), S(O)ₙ₂, CHOR³, NR³;

### X², X^{2′} independently=H, F;

n=2-6;
n′=1-6;
n²=0-2;

### A=H, OH, optionally (un)substituted aryl (substitution as defined by Z below), (un)substituted heterocycle (substitution as defined by Z below); and

### Z=H, Cl, F, Br, I, OR³, CN, OH, CF₃, R⁴, NO₂; and

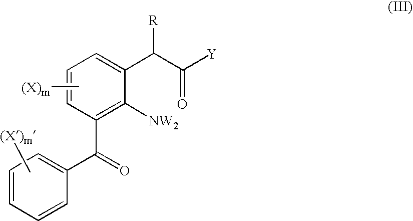

wherein

### R=H, C₁₋₄ (un)branched alkyl, CF₃, SR⁴

### Y=NR″R′;

### R′=H, C₁₋₁₀ (un)branched alkyl, (un)substituted (substitution as defined by X below), (un)substituted heterocycle (substitution as defined by X below), —(CH₂)ₙZ(CH₂)ₙ.A;

n=2-6;
n′=1-6;

### Z=nothing, O, C═O, OC(═O), C(═O)O, C(═O)NR³, NR³C(═O), S(O)ₙ₂, CHOR³, NR³;

n²=0-2;

### R³=H, C₁₋₆ (un)branched alkyl, (un)substituted aryl (substitution as defined by X below), (un)substituted heterocycle (substitution as defined by X below)

### A=H, OH, optionally (un)substituted aryl (substitution as defined by X below), (un)substituted heterocycle (substitution as defined by X below), —(CH₂)ₙOR³;

### R″=H, OH, OR′

### X and X′ independently=H, F, Cl, Br, I, OR′, CN, OH, S(O)ₙ₂R⁴, CF₃, R⁴, NO₂;

### R⁴=C₁₋₆ (un)branched alkyl;

m=0-3;
m′=0-5; and

### W=O, H.

Preferred compounds of formulas (I) and (II) are those wherein:

### R¹=H, C₁₋₄ (un)branched alkyl, (un)substituted (substitution as defined by Z below);

### R², X^{2′}, R⁴, R⁵=H;

### X²=F; and

### Z=Cl, F, Br, OH.

More preferred are compounds of formulas (I) and (II) wherein R¹=H, C₁₋₃ alkyl. The most preferred compound of formula (I) for use in the present invention is 2-(3-fluoro-4-phenyl)-propionamide. The most preferred compound of formula (II) for use in the present invention is 5-benzoyl-2,3-dihydro-1H-pyrrolizine-1-carboxamide.

Preferred compounds of formula (III) are those wherein:

### R=H, C₁₋₂ alkyl;

### R′=H, C₁₋₆ (un)branched alkyl, —(CH₂)ₙZ(CH₂)ₙ.A;

### Z=nothing, O, CHOR³, NR³;

### R₃=H;

### A=H, OH, (un)substituted aryl (substitution as defined by X below);

### X and X′ independently=H, F, Cl, Br, CN, CF₃, OR′, SR⁴, R⁴;

### R″=H;

### R⁴=C₁₋₄ (un)branched alkyl;

m=0-2;
m′=0-2;

### W=H;

n=2-4; and
n′=0-3.

The most preferred compounds of formula (III) are 2-amino-3-(4-fluorobenzoyl)-phenylacetamide; 2-amino-3-benzoyl-phenylacetamide (nepafenac); and 2-amino-3-(4-chlorobenzoyl)-phenylacetamide.

The compounds of formulas (I)-(III) are known and can readily be made by one skilled in the art. See, for example, U.S. Pat. Nos. 6,646,003 and 5,475,034, the entire contents of which are incorporated herein by reference.

The compositions of the present invention contain an anti-inflammatory effective amount of an amide derivative of a carboxylic acid NSAID. The compositions generally contain from 0.01 to 0.5% of an amide derivative of a carboxylic acid NSAID.

In addition to an amide derivative of formulas (I)-(III), the compositions of the present invention also contain a sulfite salt. Suitable sulfite salts include sodium sulfite; potassium sulfite; magnesium sulfite; calcium sulfite; sodium bisulfite; potassium bisulfite; magnesium bisulfite; calcium bisulfite; sodium metabisulfite; potassium metabisulfite; and calcium metabisulfite.

Most preferred is the sodium sulfite salt (Na₂SO₃). The compositions of the present invention comprise a sulfite salt in an amount effective to attenuate or prevent mitochondria swelling and cytochrome c release The compositions of the present invention generally comprise a sulfite salt in an amount from 0.001-0.09%, preferably 0.01-0.09%.

The compositions of the present invention also comprise an ophthalmically acceptable vehicle for topical administration to the eye. The compositions may be formulated into a variety of topically administrable ophthalmic compositions, such as solutions, suspensions, emulsions, gels or ointments. The most preferred form of delivery is by aqueous eye drops, but gels or ointments can also be used. Aqueous eye drops, gels and ointments can be formulated according to conventional technology and would include one or more excipients. For example, topically administrable compositions may contain surfactants, e.g., polysorbate 80 or tyloxapol, tonicity-adjusting agents, preservatives, buffering agents, and thickening agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 5.5-8.

Topical ophthalmic products are typically packaged in multidose form. Preservatives are typically required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically will not contain a preservative and will be unpreserved.

A representative eye drop formulation is provided below in Example 1.

### EXAMPLE 1

Topical Ophthalmic Composition

| Ingredient | % (w/v) |
|---|---|
| Nepafenac | 0.1 |
| Sodium Sulfite | 0.09 |
| Benzalkonium Chloride | 0.005 |
| Carbomer 974P | 0.5 |
| Tyloxapol | 0.01 |
| Edetate Disodium | 0.01 |
| Mannitol | 2.4 |
| Sodium Chloride | 0.4 |
| NaOH/HCl | q.s. pH 7.3–7.7 |
| Purified Water | q.s. to 100 |

### EXAMPLE 2

Topical Ophthalmic Composition

| Ingredient | % (w/v) |
|---|---|
| Nepafenac | 0.1 |
| Sodium Sulfite | 0.05 |
| Benzalkonium Chloride | 0.005 |
| Carbomer 974P | 0.5 |
| Tyloxapol | 0.01 |
| Edetate Disodium | 0.01 |
| Mannitol | 2.4 |
| Sodium Chloride | 0.4 |
| NaOH/HCl | q.s. pH 6.8–7.8 |
| Purified Water | q.s. to 100 |

### EXAMPLES 3-5

To evaluate the effects of sulfite salts on carboxylic acid NSAID-induced mitochondrial swelling, the following assay was used. Mitochondria were prepared from the livers of male Sprague Dawley rats according to the procedure of Broekemeier et al. (J. Biol. Chem 1985, 260, 105-113) Briefly, 20 g of liver were homogenized with 3 strokes in an ice-cold, iso-osmotic 3.0 mM HEPES buffer that was supplemented with 207 mM mannitol, 63 mM sucrose, 2.0 mM EGTA, and 2 mg/ml of fatty acid-free bovine serum albumin (pH 7.4). An initial low speed centrifugation (600×g for 10 minutes) was conducted to remove nuclei and cell debris. The pellet was discarded and the supernatant was centrifuged at 7,740×g for 10 minutes to obtain a crude mitochondrial pellet. The supernatant was discarded and the pellet suspended in 30 mL of ice-cold, iso-osmotic washing buffer (3.0 mM HEPES buffer containing 207 mM mannitol and 63 mM sucrose, pH 7.4). The suspension was centrifuged at 7,740×g for 10 minutes. The mitochondrial pellet was suspended in 30 mL of ice-cold wash buffer and centrifuged at 10,100×g for 10 minutes. The mitochondrial pellet was suspended in an appropriate volume of the ice-cold, iso-osmotic 3.0 mM HEPES buffer containing 207 mM mannitol and 63 mM sucrose (pH 7.4). The mitochondrial suspension was placed on ice for immediate assay. An aliquot of the mitochondrial preparation was added to a 5.0 mL cuvette (1.0 cm path length) that contained 2.95 mL of iso-osmotic HEPES buffer, supplemented with sodium succinate and rotenone. An appropriate aliquote of the mitochondrial suspension was added to the cuvette and swelling was monitored by light scattering at 540 nm for a period of 17 minutes. When drug effects were examined, mitochondria were initially exposed for a period of one minute to the test article before the addition of either buffer, t-BOOH (150 μM) or sodium sulfite (300 μM).

### EXAMPLE 3

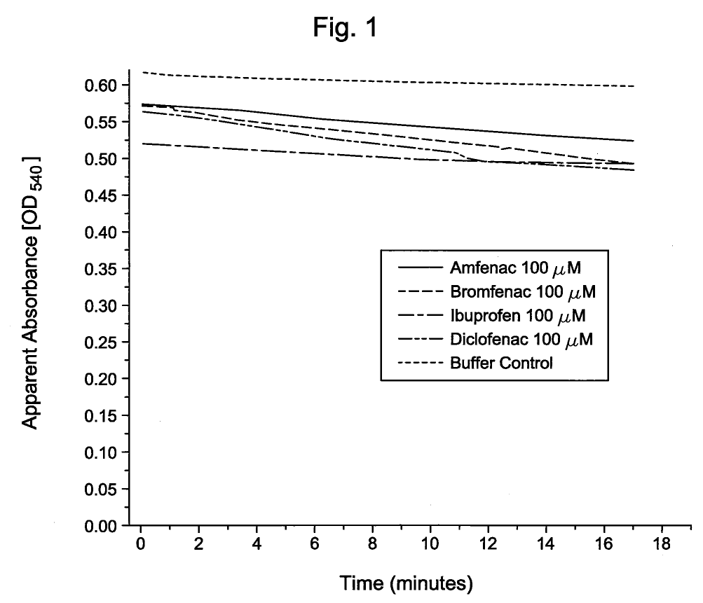

The time course of the in vitro swelling response of non-peroxide stressed (control) mitochondria following addition of a) buffer (control) b) amfenac (100 μM), c) bromfenac (100 μM), d) ibuprofen (100 μM), and e) diclofenac (100 μM) is plotted in FIG. 1. These results show that carboxylic acid NSAID's do not affect swelling of normal, unstressed mitochondria.

### EXAMPLE 4

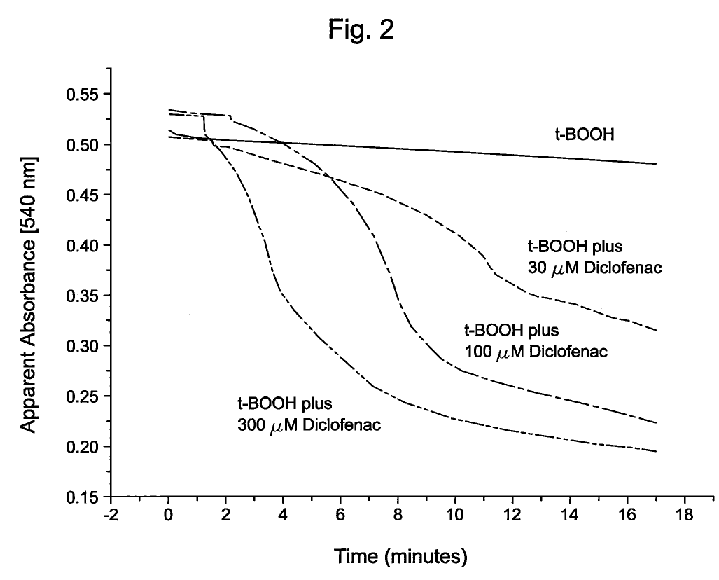

The time course of the in vitro swelling response of mitochondria following addition of t-BOOH (150 μM), or t-BOOH/diclofenac (150 μM/30 μM), t-BOOH/diclofenac (150 μM/100 μM), t-BOOH/diclofenac (150 μM/300 μM) is plotted in FIG. 2. These results show that, unlike Example 3, if mitochondria are stressed with e.g., t-BOOH, carboxylic acid NSAID's such as diclofenac promote the swelling of mitochondria.

### EXAMPLE 5

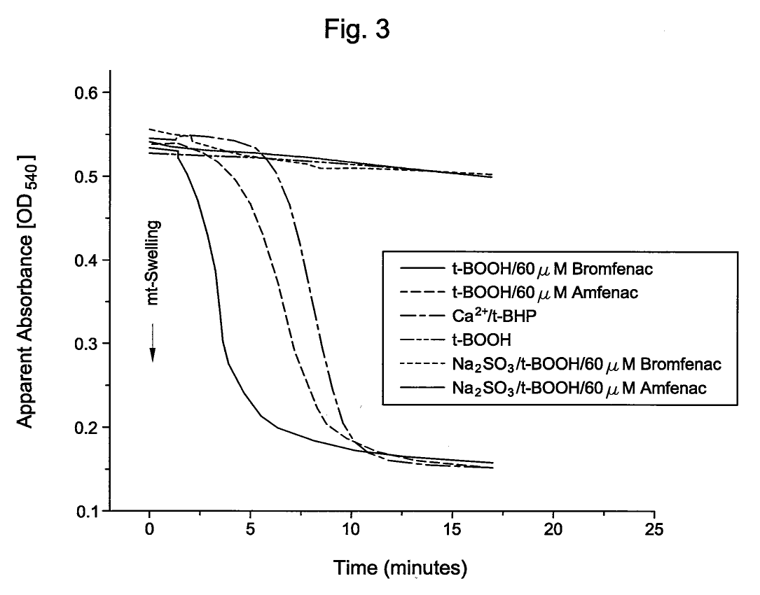

The time course of the in vitro swelling response of peroxide (t-BOOH, 150 μM)-stressed mitochondria following addition of a) 60 μM amfenac; b) 60 μM bromfenac; c) 300 μM sodium sulfite/60 μM amfenac; d) 300 μM sodium sulfite/60 μM bromfenac; e) nothing (negative control); CaCl₂ (positive control) is plotted in FIG. 3. These results show that mitochondrial swelling is prevented when a sulfite salt is added to “peroxide-stressed” mitochondria prior to addition of diclofenac.

As mentioned above, the compositions of the present invention comprise both an amide derivative of a carboxylic acid NSAID and a sulfite salt. In a preferred embodiment, the present invention also relates to a method of treating an ophthalmic inflammatory disorder, wherein the method comprises topically administering a composition comprising both an amide derivative of a carboxylic acid NSAID and a sulfite salt to the eye of a mammal in need thereof. According to another embodiment of the present invention, however, a composition comprising a sulfite salt is administered sequentially (e.g., within 10 minutes, preferably within 5 minutes, and more preferably within 2 minutes) in relation to a composition comprising an amide derivative of a carboxylic acid NSAID. In this embodiment where separate compositions are sequentially administered, the composition comprising the sulfite salt is preferably administered before the composition comprising the amide derivative of a carboxylic acid NSAID.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A topically administrable ophthalmic composition comprising
a) an amide derivative of a carboxylic acid non-steroidal anti-inflammatory agent in an anti-inflammatory effective amount, wherein the amide derivative is a compound of formulas (I)-(III): 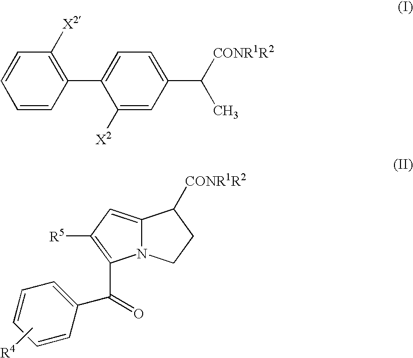
wherein for both formulas (I) and (II)
R¹=H, C₁₋₆ (un)branched alkyl, (un)substituted (substitution as defined by Z below), —(CH₂)ₙ—X—(CH₂)ₙ.A;
R²=H, C₁₋₃ alkyl, OR³;
R³=H, C₁₋₃ alkyl;
R⁴=H, Me-, MeO—, MeS—;
R⁵=H, Me-;
X=nothing (carbon-carbon bond), O, C═O, OC(═O), C(═O)O, C(═O)NR³, NR³C(═O), S(O)ₙ₂, CHOR³, NR³;
X², X^{2′} independently=H, F;
n=2-6;
n′=1-6;
n²=0-2;
A=H, OH, optionally (un)substituted aryl (substitution as defined by Z below), (un)substituted heterocycle (substitution as defined by Z below); and
Z=H, Cl, F, Br, I, OR³, CN, OH, CF₃, R⁴, NO₂; and 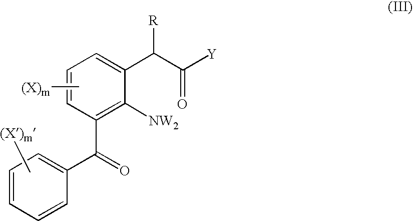
wherein for formula (III)
R=H, C₁₋₄ (un)branched alkyl, CF₃, SR⁴
Y=NR″R′;
R′=H, C₁₋₁₀ (un)branched alkyl, (un)substituted (substitution as defined by X below), (un)substituted heterocycle (substitution as defined by X below), —(CH₂)ₙZ(CH₂)ₙ.A;
n=2-6;
n′=1-6;
Z=nothing, O, C═O, OC(═O), C(═O)O, C(═O)NR³, NR³C(═O), S(O)ₙ₂, CHOR³, NR³;
n=0-2;
R³=H, C₁₋₆ (un)branched alkyl, (un)substituted aryl (substitution as defined by X below), (un)substituted heterocycle (substitution as defined by X below) A=H, OH, optionally (un)substituted aryl (substitution as defined by X below), (un)substituted heterocycle (substitution as defined by X below), —(CH₂)ₙOR³;
R″=H, OH, OR′
X and X′ independently=H, F, Cl, Br, I, OR′, CN, OH, S(O)ₙ₂R⁴, CF₃, R⁴, NO₂;
R⁴=C₁₋₆ (un)branched alkyl;
m=0-3;
m′=0-5; and
W=O, H,
b) a sulfite salt in an amount from 0.001-0.09% (w/v), and
c) an ophthalmically acceptable vehicle.

2. The composition of claim 1 wherein for formulas (I) and (II),
R¹=H, C₁₋₄ (un)branched alkyl, (un)substituted (substitution as defined by Z below);
R², R^{2′}, R⁴, R⁵=H;
X²=F; and
Z=Cl, F, Br, OH,
and
for formula (III),
R=H, C₁₋₂ alkyl;
R′=H, C₁₋₆ (un)branched alkyl, —(CH₂)ₙZ(CH₂)ₙ.A;
Z=nothing, O, CHOR³, NR³;
R₃=H;
A=H, OH, (un)substituted aryl (substitution as defined by X below);
X and X′ independently=H, F, Cl, Br, CN, CF₃, OR′, SR⁴, R⁴;
R″=H;
R⁴=C₁₋₄ (un)branched alkyl;
m=0-2;
m′=0-2;
W=H;
n=2-4; and
n′=0-3.

3. The composition of claim 1 wherein the amide derivative of a carboxylic acid non-steroidal anti-inflammatory agent is selected from the group consisting of: 2-(3-fluoro-4-phenyl)-propionamide; 5-benzoyl-2,3-dihydro-1H-pyrrolizine-1-carboxamide; 2-amino-3-(4-fluorobenzoyl)-phenylacetamide; 2-amino-3-benzoyl-phenylacetamide; and 2-amino-3-(4-chlorobenzoyl)-phenylacetamide.

4. The composition of claim 1 wherein the composition comprises 0.01 to 0.5% (w/v) of the amide derivative of a carboxylic acid non-steroidal anti-inflammatory agent.

5. The composition of claim 1 wherein the composition comprises 0.01-0.09% (w/v) sulfite salt.

6. The composition of claim 1 wherein the sulfite salt is selected from the group consisting of sodium sulfite; potassium sulfite; magnesium sulfite; calcium sulfite; sodium bisulfite; potassium bisulfite; magnesium bisulfite; calcium bisulfite; sodium metabisulfite; potassium metabisulfite; and calcium metabisulfite.

7. The composition of claim 6 wherein the sulfite salt is sodium sulfite.

8. A method of treating an ophthalmic inflammatory disorder in a mammal's eye comprising topically administering to the mammal's eye the composition of claim 1.

9. A method of treating an ophthalmic inflammatory disorder in a mammal's eye comprising topically administering to the mammal's eye the composition of claim 7.

10. A method of treating an ophthalmic inflammatory disorder in a mammal's eye comprising sequentially administering two compositions topically to the mammal's eye, wherein one of the compositions comprises a sulfite salt in an amount from 0.001-0.09% (w/v) and an ophthalmically acceptable vehicle, and the other composition comprises an amide derivative of a carboxylic acid non-steroidal anti-inflammatory agent in an anti-inflammatory effective amount and an ophthalmically acceptable vehicle, and wherein the amide derivative of a carboxylic acid non-steroidal anti-inflammatory agent is a compound of formulas (I)-(III): 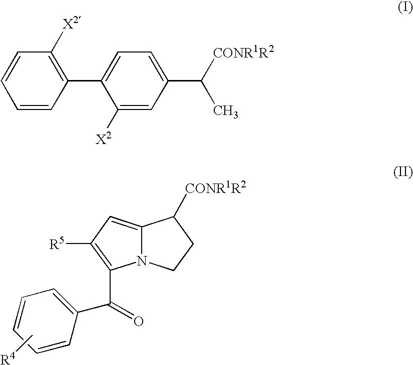
wherein for both formulas (I) and (II)
R¹=H, C₁₋₆ (un)branched alkyl, (un)substituted (substitution as defined by Z below), —(CH₂)ₙ—X—(CH₂)ₙ.A;
R²=H, C₁₋₃ alkyl, OR³;
R³=H, C₁₋₃ alkyl;
R⁴=H, Me-, MeO—, MeS—;
R⁵=H, Me-;
X=nothing (carbon-carbon bond), O, C═O, OC(═O), C(═O)O, C(═O)NR³, NR³C(═O), S(O)ₙ₂, CHOR³, NR³;
X², X^{2′} independently=H, F;
n=2-6;
n′=1-6;
n²=0-2;
A=H, OH, optionally (un)substituted aryl (substitution as defined by Z below), (un)substituted heterocycle (substitution as defined by Z below); and
Z=H, Cl, F, Br, I, OR³, CN, OH, CF₃, R⁴, NO₂; and 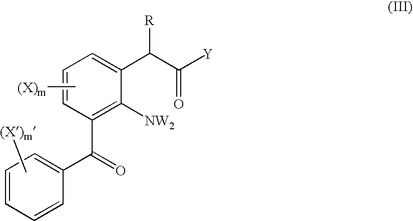
wherein for formula (III)
R=H, C₁₋₄ (un)branched alkyl, CF₃, SR⁴
Y=NR″R′;
R′=H, C₁₋₁₀ (un)branched alkyl, (un)substituted (substitution as defined by X below), (un)substituted heterocycle (substitution as defined by X below), —(CH₂)ₙZ(CH₂)ₙ.A;
n=2-6;
n′=1-6;
Z=nothing, O, C═O, OC(═O), C(═O)O, C(═O)NR³, NR³C(═O), S(O)ₙ₂, CHOR³, NR³;
n²=0-2;
R³=H, C₁₋₆ (un)branched alkyl, (un)substituted aryl (substitution as defined by X below), (un)substituted heterocycle (substitution as defined by X below) A=H, OH, optionally (un)substituted aryl (substitution as defined by X below), (un)substituted heterocycle (substitution as defined by X below), —(CH₂)ₙOR³;
R″=H, OH, OR′
X and X′ independently=H, F, Cl, Br, I, OR′, CN, OH, S(O)ₙ₂R⁴, CF₃, R⁴, NO₂;
R⁴=C₁₋₆ (un)branched alkyl;
m=0-3;
m′=0-5; and
W=O, H.

11. The method of claim 10 wherein the composition comprising the sulfite salt is administered before the composition comprising the amide derivative of a carboxylic acid non-steroidal anti-inflammatory agent.

12. The method of claim 10 wherein the sulfite salt is selected from the group consisting of sodium sulfite; potassium sulfite; magnesium sulfite; calcium sulfite; sodium bisulfite; potassium bisulfite; magnesium bisulfite; calcium bisulfite; sodium metabisulfite; potassium metabisulfite; and calcium metabisulfite.

13. The method of claim 10 wherein the amide derivative of a carboxylic acid non-steroidal anti-inflammatory agent is selected from the group consisting of: 2-(3-fluoro-4-phenyl)-propionamide; 5-benzoyl-2,3-dihydro-1H-pyrrolizine-1-carboxamide; 2-amino-3-(4-fluorobenzoyl)-phenylacetamide; 2-amino-3-benzoyl-phenylacetamide; and 2-amino-3-(4-chlorobenzoyl)-phenylacetamide.
